(19) United States
(12) Patent Application Publication
(10) Pub. No.: US 2005/0118704 A1
(43) Pub. Date: Jun. 2, 2005

(54) System and method for real-time detection and remote monitoring of pathogens

(76) Inventor: Brana Malobabic, St. Laurent (CA)

Correspondence Address: FOGG AND ASSOCIATES, LLC, P.O. BOX 581339, MINNEAPOLIS, MN, 55458-1339, US

(21) Appl. No.: 10/746,936

(22) Filed: Dec. 24, 2003

Related U.S. Application Data
(60) Provisional application No. 60/437,041, filed on Dec. 31, 2002.

(51) Int. Cl. C12M 1/34

(52) U.S. Cl. 435/287.1

(57) ABSTRACT

A real-time continuous detector device for detection of contaminants in a sample includes at least one sample management module, including a mechanism to control sample flow management to place the sample on a sample area. At least one electronic module for data processing and control is provided, and there is at least one optical module consisting of at least one real-time replaceable sensor cartridge containing a plurality of sensors, and at least one real-time optical pathogen detector connected to the electronic module for data processing. Power is provided and the device is equipped with at least one secure communication module adapted to transmit encrypted information over a secure link to a remote location and for receiving information. The invention also discloses a system making use of detectors for real-time detection of contaminants and for early warning capability, among others.

### CROSS REFERENCE TO RELATED APPLICATIONS

This application is related to and claims the benefit of the filing date of co-pending provisional application Ser. No. 60/437,041 (the “'041 Application”), filed on Dec. 31, 2002. The '041 Application is incorporated by reference.

### FIELD OF THE INVENTION

The present invention concerns a system for the real-time detection and monitoring of pathogens.

### DESCRIPTION OF THE PRIOR ART

The problem to which the present invention is directed is described in detail on a water quality example which follows. Similar problems and prior art exists in air monitoring, food safety, healthcare (early infection detection—humans and animals) and anti-terrorist readiness. There are attempts to resolve those problems, but an effective solution is not commercially available.

### WATER QUALITY MONITORING EXAMPLE

Water-borne pathogens in industrial, domestic, recreational and potable water systems can pose serious health risks to consumers. The protozoan Cryptosporidium and the bacteria E. Coli, Giarda, and Salmonella are some common water-contaminating micro-organisms that can cause diarrhoea, dysentery, hepatitis, cholera, and typhoid fever, and in severe cases, can even be fatal. The Walkerton tragedy in 2000 attracted a lot of media attention in Canada as seven individuals died due to an E. Coli outbreak in the municipal water supply. However, the scope of the problem is much larger than that, with biological contaminants causing about 900,000 illnesses and killing about 900 people per year in the US alone (http://www.bergen.org/AAST/Projects/ES/WS/pollution).

Our public water infrastructure and waterways are vulnerable to threats, either intentional or accidental, and governments are increasingly becoming aware of the urgent need to improve the secure supply of drinking water to consumers. Although regulations do exist to monitor the quality of drinking water, the testing methods and, more importantly, the rigour in applying these methods are not always reliable.

The detection and identification of bacterial and viral pathogens as well as general contaminants in water supplies continues to rely on conventional culturing techniques. These techniques typically require a few days in order to obtain the correct results, failing to alert users/authorities to quality control problems until well after the fact.

Many viruses, bacteria and protozoa can go undetected in our drinking water supply with water contamination only being reported two to eight days after the event. The process of testing for pathogens in water is entirely a manual process, therefore an extremely costly one.

Pathogen intrusion can be:

Pollution caused by man/animals in source or groundwater water.

Terrorist intrusion.

Natural disaster.

Water infrastructure faults (water leakage due to aging water infrastructure, open storage reservoir, improper disinfecting, hazardous process fluids in user facilities, etc).

Existing treatment technologies allow water treatment plant operators to inactivate or remove many chemical and/or biological contaminants. However, there is no monitoring readily available that allows utility operators to prevent contaminants from entering the homes. Thus, micro-organisms can enter the plant and eventually reach the customer.

An emerging opportunity is to carry out microbial audits at water treatment plants, individual buildings and natural water reserves. Apart from providing data for statutory compliance, these audits could help to identify weak links in the treatment chain, devise treatment strategies to target harmful microbes and carry out complete risk assessment of the water systems.

Drinking water quality management decisions are based on the information produced by source water quality monitoring, drinking water quality monitoring and waterborne disease surveillance. At present, there is no surveillance system that has all three of these components and makes that information available in real-time. Such system would improve our capacity to understand, predict and prevent future problems with water such as outbreaks of infectious disease caused by contaminated drinking water—the Cryptosporidiosis outbreak in Milwaukee and the E. coli O157:H7 outbreak that occurred in Ontario (2000). Recent outbreaks of waterborne disease have heightened awareness of the fact that threats to water quality can have a profound impact on their health, the environment and the economy.

The World Health Organization (WHO) has described a hypothetical contamination scenario for a city of 50,000 people with a daily water use of 400 L per person per day. Each person drinks 0.5 L of water per day, while a lethal dose of a given toxin is 1 μg, according to the scenario. The total dose required to contaminate 20 million L of water is 40 g, assuming a homogeneous solution. Allowing a factor 6 to compensate for unequal distribution and dilution, the total amount of toxin required would be 24 Og. The toxin would have to be delivered over a period of time, most likely from midnight to 6 am. The affected population would show effects about 8 hours later, or around the middle of the afternoon.

Other issues with the current water analysis are that very small samples of water are analyzed infrequently (for example 100 ml every week, every second week or even every fourth week) and water quality process control relies on those results, while thousand of liters of water are reaching consumers.

Very few contaminants are monitored as part of the water quality procedures. For example, some viruses are non-culturable, and have extensive testing costs, and therefore regulatory procedures omit them.

The American Water Works Association examined water utilities and determined that $250 billion is needed over the next 30 years to replace its aging infrastructure. The aging water distribution infrastructures need to have their water quality issues addressed at the same time.

Some contaminants are chlorine-resistant, such as anthrax, which if introduced in source water (we do not currently monitor for anthrax presence) or in a water distribution system could cause death. There are many other contaminants, which are chlorine-resistant and are widely accessible.

Chemical disinfectants (such as chlorine) are added to water supplies for microbiological protection. A major challenge for water suppliers is how to balance the risks from microbial pathogens and disinfection byproducts (DBPs). Chlorine reacts with NOM (natural organic matter) to form DBPs that are considered to be of public concern (as they might cause cancer in the long term).

Technologies such as PCR and immunoassay improved testing time (1 hour possibly), but manual device operating, add-on reagents, in some cases sensitivity issues, interference in a real-water flow problems and skilled personnel required to handle tests makes those methods non-suitable for automatic real-time monitoring of pathogens.
Present costs of water quality monitoring systems are enormous:
Loss of peoples lives due to lack of information and late decisions.
Cultural samples testing.
Public awareness concern, lawsuits.
Technicians'salaries and process of taking and delivering samples to labs.
Certified lab costs, labs maintenance, inspection, reporting and review.
Healthcare costs due to waterborne diseases.
Current Challenges of water operators are:
Long delays in reporting water quality (i.e. bacterial) test results.
Currently detecting limited number of known contaminants.
Current tests rely on conventional culturing techniques.
Non-automated costly process and systems.
Time consuming, trained technicians.
Increased threat of pollution by mankind.
Aging water infrastructure.
Bio-terrorism threat to our water supplies.
   Natural disaster threats.
Confidence in our infrastructure, public concerns.
Ever increasing list of contaminants.
   Short Description of Pathogen Management in Other Fields
   Food Safety Problems

The cost of healthcare caused by food borne illnesses in North America is larger than $1 billion. The food processing industry annually carries out more than 144 million microbial tests costing $5 to $10 each. About 24 million of these tests are for detection of food pathogens based on biochemical profile analysis, immunogenic tests and DNA/RNA probes, which take at least a few hours or days.

Human and Animal Health

In remote areas, old people, kids etc. might have a lack of a medical care, especially in emergency situations. In addition, bacterial infections have become an increasing health problem because of the advent of antibiotic-resistant strains of bacteria. Further, individuals in developing countries who may be malnourished or lack adequate sanitary facilities may also support a large amount of opportunistic bacteria, many of which may cause sickness and disease.

In veterinary medicine, livestock living in close quarters also may be prey to infections caused by a variety of different types of microbes.

Thus, there is a need to timely react on microbial infections in humans and animals. The present invention allows users to get full analysis on possible bacterial, viral infections and other diseases within minutes, making sure that information reaches health-care professionals in a timely manner, and can be viewed by a user or an authorized health care professional only.

There is thus a need for a system which automatically and in real-time makes pathogen measurements, operates with little or no human intervention, with high sensitivity and specificity (even 1 microorganism/ml), recognizes live vs. dead pathogens and communicates information safely in real-time to users. There is also a need to quickly process results and recommend hazard response measures. Failure to have results in real-time might have severe consequences in some cases even deaths. Such systems should preferably be able to self-calibrate, learn from previous results and minimize false positives and negatives in order to function efficiently.

Individuals who wish to check their water, air, food or health have a need for miniaturized hand-held units, to test for presence and concentration of pathogens in real-time.

### SUMMARY OF THE INVENTION

It is an object of the present invention to provide a system for the real-time detection and monitoring of pathogens. In accordance with the invention, this object is achieved with a real-time continuous detector device for detection of contaminants in a sample, comprising:
at least one sample management module, including means to control sample flow management to place said sample on a sample area;
at least one electronic module for data processing and control;
at least one optical module consisting of at least one real-time replaceable sensor cartridge containing a plurality of sensors, and at least one real-time optical pathogen detector connected to said electronic module for data processing;
at least one power module; and
at least one secure communication module adapted to transmit encrypted information over a secure link to a remote location and for receiving information.

This object is further achieved with an automated process for real-time monitoring of contaminants with early warning capability comprising:
a plurality of detectors; and
a central location for receiving information from said detectors,
for real-time detection and quantity of contaminants;
for real-time results processing and secure transmission;
for secure storage and pathogen information processing;
for early warning capability and contaminant path prediction; and
for hazard response measures.

Another aspect of the invention is achieved with a system for real-time monitoring and detection of pathogens comprising:
a plurality of real-time detector devices for detection and monitoring of pathogens, each of said devices comprising:
a power module;
a sensor module including at least one sensor;
a real-time detector module;
computer means for processing data produced by said real-time detector module; managing power; and managing communications;
a secure communications module for communicating data to and from a remote location; and
a central monitoring station for receiving data from said devices, said central monitoring station including means for analyzing said data and generating alarm conditions upon detection of a pathogen.

In broad terms, the present invention is an improved system for automated real-time detection and monitoring of pathogens with hazard prevention measures. The object of this invention is to provide a self-operated pathogen monitoring system, that can perform quantitative and qualitative analysis of many substance types, and has the capability to quickly make decisions based on learned information. Additionally, mobile hand-held units are preferably connected to the system to perform field-testing. The devices are housed in enclosures, where the various components are modular and accessible for maintenance and serviceability.

A variety of hardware features have been incorporated into the continuous devices, including redundancy, modularity, pre-concentration filters for greater sensitivity and analyzing larger volumes of substance, self-cleaning storage, new and used sensor cartridges, self-moving sensors for extended device usage, automatic flow management, detectors for measuring parameters or change in optical signals, processors and secure communication modules which only upon secure authentication and secure hand-shake protocol can transfer the results to a remote storage.

One embodiment of the invention includes a digital signal processor for false positives (and false negatives) reduction and sensor control operation—detection of specific pathogens, amount of test times detecting the same pathogen. This feature improves accuracy to up to 99%.

The system is aware of unit geographical positions, previous results and all unit results, which represents a source of information for prediction capabilities.

### DESCRIPTION OF THE FIGURES

The present invention will be better understood after reading a description of a preferred embodiment thereof, made in reference to the following drawings in which:

FIG. 1 is a schematic representation of the system according to a preferred embodiment of the present invention;

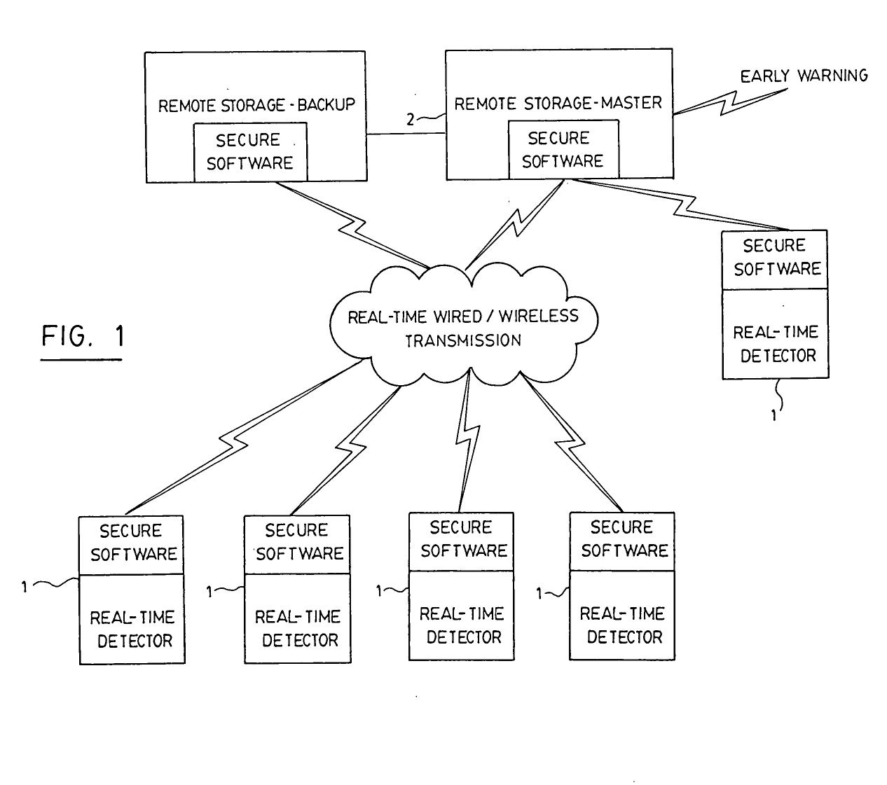

FIG. 2 is a schematic representation of the continuous real-time detection unit according to a preferred embodiment of the present invention; and

FIG. 3 is a schematic representation of an external and internal view of the hand-held real-time detection unit according to a preferred embodiment of the present invention.

### DESCRIPTION OF A PREFERRED EMBODIMENT OF THE INVENTION

In the context of the present invention, “Real-time detection” means direct pathogen detection in less than 30 minutes.

In its preferred embodiment, the invention represents real time pathogens detection (bacteria, viruses and protozoan) in substances (liquids, gases and other matter).

The system can be used for environmental monitoring (water and air), food safety, life sciences (early infection detection in humans and animals) and anti-terroist readiness among others.

Besides pathogens, the system can include other (secondary) real-time contaminant detectors:
Chemicals (mercury, arsenic, sulfur, mustard, etc.).
Biochemical Toxins (Ricin, Microcystin, Teradotoxin, etc).
War agents (nerve, blood, choking, blister agents).
Radioactive Material (uranium-238, iridium-192, strontium-90, cobalt).

Benefits of this invention are shown in a water quality-monitoring example which follows. However, these benefits in general transcend water applications and are relevant and applicable to food, air, life sciences and other applications.

The advantage of a new process described by this invention are the following: it permits to obtain results faster, potentially preventing pathogens to reach users; it is an integrated information system which allows to define connections between sources of contamination and diseases; the quality process can be monitored efficiently, minimizing the impact of human mistakes; in some cases larger volumes of a given substance can be analyzed; the process is automatic and; it provides an early warning preventing hazards to occur.

The following are common water supply elements:
Water source: surface water (lake, reservoir, river), groundwater.
Transmission systems: tunnels, reservoirs, pumping facilities, storage facilities.
Distribution system: portable water through pipes to consumers.

Water is supplied from reservoirs, rivers, lakes or groundwater.

More effective water quality monitoring solutions must be cost-effective, provide continuous monitoring and early warning.

This invention benefits (example, in water quality monitoring):
End-to-end water quality monitoring solution.
Real-time detection of pathogens in water.
Immediate availability of test results.
24×7 on-line access to water quality information.
Early warning capability to prevent Walkerton-like events.
Contamination path prediction.
Hazard prevention measures.
Can operate with no user intervention.
Fully secure critical information collection and distribution system.
Larger number of contaminants detected than by traditional methods.
Larger water volumes analyzed and greater sensitivity achieved than by traditional methods.
Greater cost efficiency than the current traditional process.
Integration capability of systems for source and drinking water.
Statistics capability for echo-system monitoring.
Detecting water infrastructure faults by monitoring level of contaminants.

Real-time detectors can be installed in reservoirs, rivers, lakes, groundwater, water treatment plants, water pipes, businesses, swimming pools, wells, oceans, healthcare environments such as hospitals, homes and/or carried by individuals.

This invention allows water operators to understand timely water quality information, so they could minimize DBPs and electrical power consumption required to run disaffection processes. Most importantly, it provides an early warning of contamination that can occur in water supplies, preventing major and possible epidemic risks from spreading, threatening the nation's human and animal life.

The present invention reduces water quality testing costs by timely recognition, analysis and notice of deviations in the integrity of water supplies. It can pinpoint source contamination and, predict contamination path in geographical clusters of water supplies.

The system can also collect centralized information on water, air, food, human and animal pathogen presence to safeguard nations and world health in general.

System Components

The present invention represents an automated system, where hundreds of remote smart detectors are monitored and controlled in real-time. This system provides not only precise measurements, but also completely modular detectors in any environment. Through a systematic analysis of contaminant indicators, the systems makes effective use of the results and forms a decision-making tool with prediction capabilities. It has the capability to direct on-line users, through an extensive definition and implementation information on hazard response measures.

The present invention can be used as a continuous water quality monitoring system, which detects presence of one or multiple pathogens in water in real-time.

A network of intelligent real-time detectors 1, automatically detects, collects and analyzes information related to contaminants. Intelligent networks of detectors 1 transmit securely real-time contaminant information to a remote central storage 2. Each contaminant detector 1 has the capability to communicate with neighboring detectors 1 (when within communication reach) and at least one remote storage system 2.

The system is adapted to perform a self-health check, and reports automatically any defects.

Real-Time Detector Device

Automatic Sampling Management Module (ASM)

In the case of a hand-held unit 1′ the substance sample is deposited onto the device, and it does not require an ASM module.

The ASM module automatically takes the substance sample. Integrated pumps 112 control the substance flow, including but. not limited to substance entry, path and removal by receiving instructions from a processor. The walls of the ASM module, in case of water monitoring, are made of Teflon or other suitable materials, which minimize the biofilm growth.

The liquid preferably flows though a filter 107, which is used for pre-concentration of contaminants, such that large volumes of samples can be transmitted through the filter 107, but a small concentrated temperature-controlled substance volume may be isolated and analyzed for presence or absence of contaminants. Isolated pre-concentrated volume improves sensitivity of a system, and larger volumes of substance can be monitored than by traditional methods. In some cases, the pre-concentration and sample processing step is not required. ASM has alternative substance flow paths. The pre-concentrated substance sample (or substance flow) is put into a sensor module.

Substance sampling periods are programmable. For example, it could be continuous substance flow, or every minute, every hour sampling etc. The ASM module has modular, redundant substance channels and filters. A preferred embodiment of this invention suggests continuous testing.

The ASM module therefore consist of sample entry 105 and sample processing/filtering 107. An ASM module receives instruction from processor about sample flow management from entry point to substance removal point.

The ASM module advantageously includes a replaceable cleaning substance storage 101, 103, which can be used for self-cleaning and/or sensor cleaning. The ASM module further advantageously includes a temperature control module 113 for cooling or heating of a sample, which further improves sensitivity of the system by stabilizing measurement conditions in the optical module.

Sample preparation in preparation areas 109, 111 may also be used in order to achieve greater specificity.

Optical Module

The Optical Module consists of Real-Time Sensor 119 and Real-Time Detector modules 121.

Real-Time Sensor Module

The Sensor Module 119 contains at least one sensor which can detect the presence/or absence of one or more pathogens in real-time. Sensors are small in size and can detect presence and quantity of pathogens.

The sensors are initially stored in a clean sensor cartridge 115. Sensors are coated with receptors (such as antibodies, artificial receptors or other kind and/or combination of coating chemistries), which interact with pathogens and allow for detection of presence/absence of a specific pathogen or pathogen group and/or quantity of this pathogen. Sensors detecting a group of pathogens and specific live or dead pathogen quantity can work in parallel. A detection event occurs when the target pathogen contacts the sensor surface where it interacts with one or more receptor elements. A single sensor can measure one or more pathogens. One or more sensors are active at a time.

Sensors are replaced automatically when their lifetime ends. Sensor lifetime is known or calculated by following the amount of time that the sensor was in a contact with a substance, concentration of contaminant detected by a specific sensor and a sensor-health detection system response. Periodically, sensor-health is checked by signal processing functions and self-test functions. Some sensors can be regenerated and reused within the system, and some are put in a used sensor cartridge 117. The sensor cartridges 115 and 117 can be replaced when empty.

The sensor cartridges 115, 117 receive instructions from a processor, for controlling the number of active sensors, measurements, which specific measurement and self-moving instructions.

It should be noted that sensor cartridges may have different shapes: a straight shape, a roller-like shape, etc.

According to a preferred embodiment of the invention, the sensors are small, and the sensor module includes means for precisely aligning the sensors to the detector.

The system of the present invention can use other sensing mechanisms, such as nanosensors. For example, small nanosensors, which identify microorganisms by producing the image of a specific microorganism and the resultant information is a digital image (in an amplified form), which further can be processed to produce real-time contaminant detection results.

Optionally, sensors detect chemicals, toxins and other contaminants.

Real-Time Detector Module

Real-time detector 121 measures optical signal parameters, or signal change (for example, intensity, wavelength, phase or other), processes the signal and provides detection indicators in minutes. Those detectors also quantify the number of measured contaminants present on the sensor surface. The signal to noise ratio is preferably minimized to achieve greater sensitivity.

Real-time detectors 121 provide detection without the use of conventional lab culturing and are able to operate remotely with no user intervention. The detection systems reduce the time required for biological analysis from many hours or days, to minutes.

The real-time detectors are able to self-calibrate or can be remotely calibrated.

Processing Module

The results 123 of the detector 121 are processed in processing module 70. Greater sensitivity and minimization of false positives (and false negatives) is achieved through appropriate logic, digital signal processing, communication and learning capabilities. The resultant information is digitized.

The processor manages all the robotic (automated) functions of the device 1.

The processor 70 has an optional capability to record and attach voice and video results, documenting the analysis process, environment, test sample, etc.

Self-Protection Module (SPM)

The device 1 has a self-protection mechanism 40, which prevents unauthorized users and intruders from copying data or technology. It recognizes and records tampering events—information or a physical device tampering.

Power Management Module

Power could be supplied to the unit through an AC/DC plug, solar panel, sent wirelessly to the unit, batteries or a combination thereof and the power is managed by module 60.

Secure Communication Module (SCM)

Results are secured before transmission in a secure communications module 30. A Data transmission session (can be wireless or wireline) starts when the device is authorized through the use of strong authentication and hand-shake protocols. Smart SCMs 30 have the capability to communicate with neighboring devices 1 and a central storage 2. They have the capability to learn about results of other detectors, and predict what their measurement results should be. The SCMs 30 may send a message to sensor modules to dynamically change target pathogen to be tested. Unauthorized processes and users are rejected, detected and recorded. The SCM is preferably provided with a physical positioning system chip such as a GPS.

Some results can be temporarily or permanently stored in memory 50.

Mechanical Casing

The housing is preferably modular and flexible to accept new sensors and detectors. The casing is water-resistant.

Devices have an optional camera module to record any tampering and warn users.

Real-Time Transmission Link

Real-time contaminant detection information is encrypted, securely transmitted to a database in real-time, locked and backed up. The system rejects and detects all unauthorized processes and users.

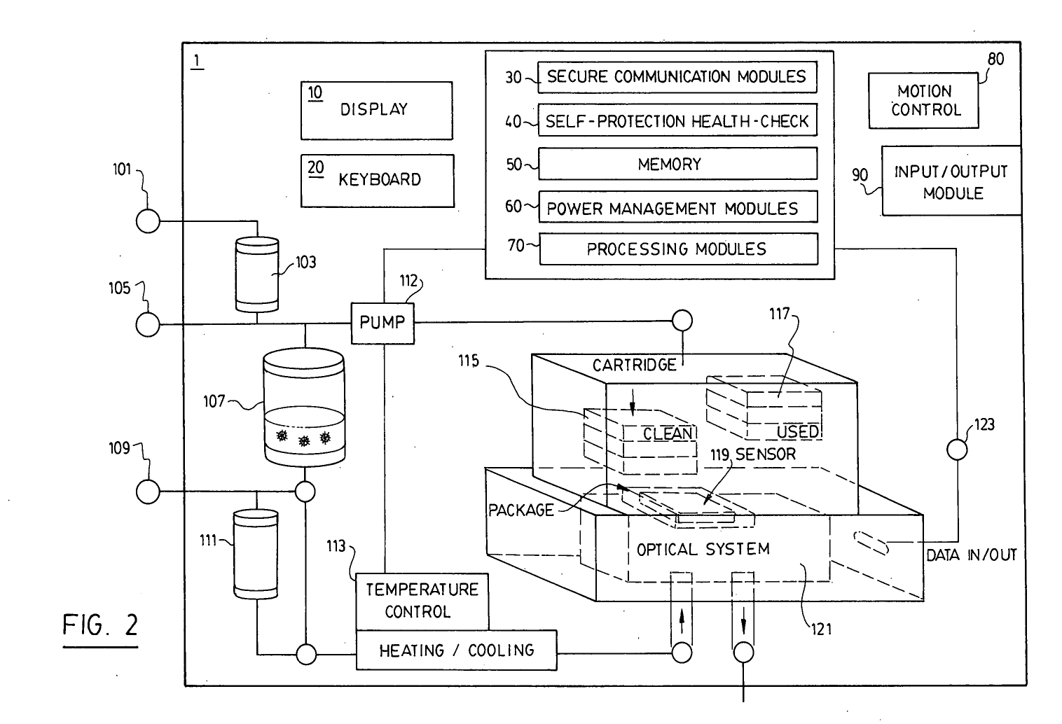

As best seen in FIG. 2, the devices preferably include a display 10 and keyboard 20, and those devices that are in water can be equipped with a motion controller 80 to move the device. Of course, input/output means 90 are also provided, but can be integrated in the SCM 30.

Remote Storage

The remote storage 2 represents one or many remote servers collecting information from remote contaminant detectors, analyzing the information and representing the information in a user-friendly form.

If critical information crosses a pre-defined threshold, an alarm is initiated (could be at the level of real-time detectors and/or remote storage). Depending on issue severity, corrective actions are initiated automatically (such as inform authorities, users, close the water distribution path, etc). Based on collected information, remote control initiates corrective actions and generates instructions to authorized personnel on hazard response measures.

Remote storage 2 collects information, systematically analyzes the information and makes prediction of contamination path, based on learned information, position of detectors, system health-check and pre-programmed mathematical formulas. Statistics are a source of information, which is used for further system learning.

Remote storage 2 has the capability to analyze results and if a hazard occurs or it can occur, the system can direct authorized users on hazard response measures.

Graphical User Interface

The Graphical user interface has a viewing capability in geographical clusters, and capability to show forecasted contamination path, for a specific pathogen (contaminant). Only authorized users can view results on-line using the graphical user interface.

Geographical Clusters

In cases of water monitoring, geographical clusters of water supplies include source water, groundwater, water treatment plants, water wells, swimming pools, distribution system, homes and other sources of water.

Geographical clusters of pathogen detection in air, food, human and animal health surveillance systems may be connected in a single system. This allows for gathering information on the cause and source of contamination.

As mentioned previously, the system may have a Motion Control Module 80 for a remote motion control, which would allow device mobility and taking multi-site samples with a single device. Images of remote areas (camera enabled) may be transmitted to a remote storage, which operator may use to navigate devices in addition to geographical maps integrated into the Graphical User Interface. Continuous devices may be capable of floating in the direction of current in case of water monitoring.

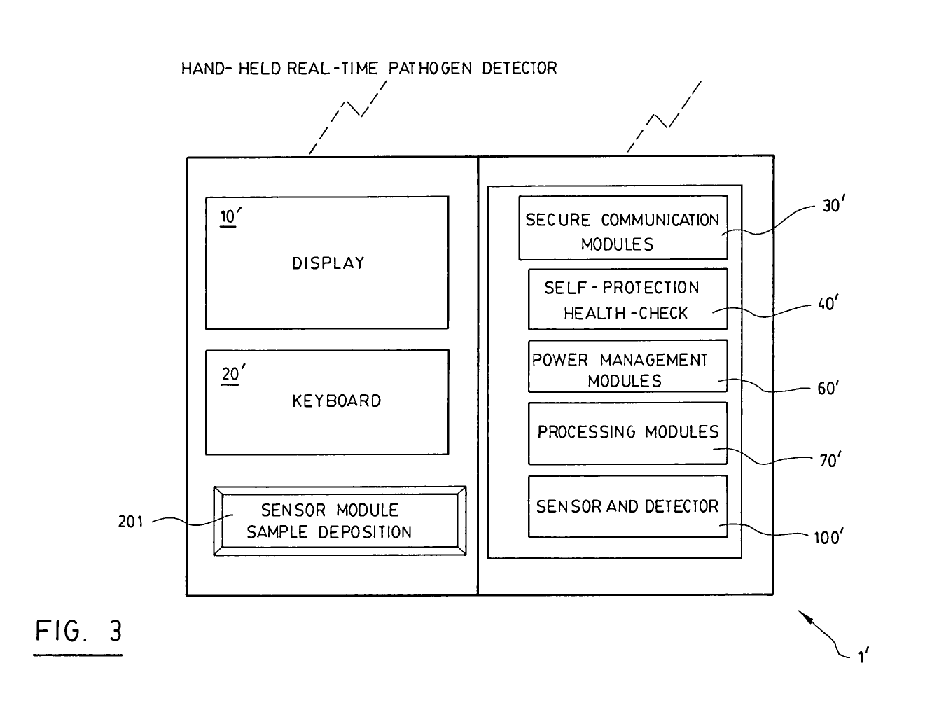

The system of the present invention also preferably includes at least one hand-held detector 1′, shown in FIG. 3.

The detector 1′ has essentially the same SCM 30′, self protection/health check 40′, power 60′, processing 70′ and sensor and detector 100′ modules as detector 1, as well as display 10′ and keyboard 20′. The major difference lies in the fact that the sample is manually deposited on the sensor 201, which has a limited life and must be manually replaced.

Although the present invention has been explained hereinabove by way of a preferred embodiment thereof, it should be pointed out that any modifications to this preferred embodiment within the scope of the appended claims is not deemed to alter or change the nature and scope of the present invention.

1. A real-time continuous detector device for detection of contaminants in a sample, comprising:
at least one sample management module, including means to control sample flow management to place said sample on a sample area;
at least one electronic module for data processing and control;
at least one optical module consisting of at least one real-time replaceable sensor cartridge containing a plurality of sensors, and at least one real-time optical pathogen detector connected to said electronic module for data processing;
at least one power module; and
at least one secure communication module adapted to transmit encrypted information over a secure link to a remote location and for receiving information.

2. A real-time detector device according to claim 1, wherein said remote location is a central monitoring station.

3. A real-time detector device according to claim 1, wherein said device further includes a camera for recording events and for taking images of each of said at least one sensor.

4. A real-time detector device according to claim 1, wherein said device detects at least one pathogen presence/absence and/or concentration in less than 30 minutes after said sample reaches said sensing area.

5. A real-time detector device according to claim 1, further comprising an automated sampling module which manages sample flow and pre-processing of said sample through said device.

6. A real-time detector device according to claim 5, further comprising redundant and alternative substance flow paths, and at least one pump.

7. A real-time detector device according to claim 5, wherein said device contains a pre-concentration filter for isolating contaminants in a smaller volume, a cleaning storage and a sample preparation storage.

8. A real-time detector device according to claim 5, wherein said device further includes a temperature controller for controlling temperature of said sample prior to deposition on said sample area.

9. A real-time detector device according to claim 5, wherein said automated sampling module comprises walls suitable for a particular substance flow which minimizes water clogs in case of water quality monitoring.

10. A real-time detector device according to claim 5, wherein said sampling module, opening and closing sample flow paths, pump actions, alternative sample paths and sampling frequency is controlled by said electronic module.

11. A real-time detector device according to claim 1, wherein said optical module for real-time detection of contaminants comprises a real-time sensor module and real-time detector module.

12. A real-time detector device according to claim 1, wherein said plurality of sensors are movable between an unused sensor area, said sample area, and a used sensor area.

13. A real-time detector device according to claim 1, wherein said sensors are coated with receptors which interact with contaminants and allow for detection of presence/absence of a specific contaminant or contaminant group and/or quantity of said contaminant.

14. A real-time detector device according to claim 1, wherein said sensors are adapted to detect one or more pathogens and other contaminants.

15. A real-time detector device according to claim 1, wherein said sensors have a sensor lifetime which is known or is determined by analyzing an amount of time that the sensor was in a contact with a substance, concentration of a contaminant detected by a specific sensor and a sensor-health detection system response.

16. A real-time detector device according to claim 1, further comprising a real-time detector module which measures optical signal parameters, or signal change and processes the signal to provide detection indicators.

17. A real-time detector device according to claim 1, wherein said power module manages at least one power supply as AC/DC, solar, self-generated power, power sent wirelessly to the unit, batteries or a combination thereof.

18. A real-time detector device according to claim 1, wherein said device is enclosed in a mechanical casing.

19. An automated process for real-time monitoring of contaminants with early warning capability comprising:
a plurality of detectors; and
a central location for receiving information from said detectors,
for real-time detection and quantity of contaminants;
for real-time results processing and secure transmission;
for secure storage and pathogen information processing;
for early warning capability and contaminant path prediction; and
for hazard response measures.

20. The process according to claim 19, wherein said detectors are selected from the group consisting of continuous detectors and hand-held detectors.

21. The process according to claim 19, further comprising the step of initiating automatic control functions to prevent hazards to occur based on learned contaminant information.

22. The process according to claim 19, further comprising the step of detecting at least one pathogen among other contaminants.

23. An automated system for real-time detection and remote monitoring of contaminants with early warning capability comprising:
at least one real-time contaminant detector;
at least one secure real-time transmission link;
at least one remote, secure storage; and
means for early warning, contamination path prediction and hazard response measures based on information received from said at least one detector.

24. A real-time detector device according to claim 23, wherein at least one of said at least one detector of a real-time contaminant detector, detects at least one pathogen presence/absence and/or concentration in less than 30 minutes after a sample reaches optical module.

25. A real-time detector device according to claim 23, wherein encrypted information is transmitted over a secured transmission link.

26. A real-time detector device according to claim 23, wherein said remote storage collects information, systematically analyzes information and makes prediction of contamination path, based on learned information, position of detectors, system health-check and pre-programmed mathematical formulas.

27. A real-time detector device according to claim 26, wherein said system initiates corrective actions automatically and generates instructions to authorized personnel on hazard response measures.

28. A real-time detector device according to claim 23, wherein said remote storage has a graphical user interface viewing capability comprised of geographical clusters, which shows contaminant levels, historical statistics, alarms if critical contaminant quantity threshold is reached and a forecasted contamination path, for a specific contaminant.

29. A real-time detector device according to claim 23, wherein at least one of said at least one detector is a real-time portable hand-held detector device for detection and monitoring of contaminants, said detector comprising:
at least one sample deposition module;
at least one optical module, which consists of at least one real-time replaceable sensor cartridge, and at least one real-time optical pathogen detector connected to an electronic module for data processing;
at least one electronic module for data processing and control;
at least one power module; and
at least one secure communication module with a capability to transmit encrypted information over a secure link to a remote storage and to receive information.

30. A real-time detector device according to claim 29, wherein said portable hand-held detector detects at least one pathogen presence/absence and/or concentration in less than 30 minutes after a sample reaches said optical module.

31. A real-time detector device according to claim 29, wherein said sensors are coated with receptors, which interact with pathogens and allow for detection of presence/absence of a specific contaminant or contaminant group and/or quantity of this contaminant.

32. A real-time detector device according to claim 29, wherein said detector is adapted to detect contaminants.

33. A system for real-time monitoring and detection of pathogens comprising:
a plurality of real-time detector devices for detection and monitoring of pathogens, each of said devices comprising:
a power module;
a sensor module including at least one sensor;
a real-time detector module;
computer means for processing data produced by said real-time detector module; managing power; and managing communications;
a secure communications module for communicating data to and from a remote location; and
a central monitoring station for receiving data from said devices, said central monitoring station including means for analyzing said data and generating alarm conditions upon detection of a pathogen.

34. A real-time detector according to claim 1, wherein said remote location is another real-time detector device.
